(19) United States
(12) Patent Application Publication
(10) Pub. No.: US 2011/0178408 A1
(43) Pub. Date: Jul. 21, 2011

(54) Accurate Low-Cost Non-Invasive Body Fat Measurement

(75) Inventor: Robert D. ROSENTHAL, Hagerstown, MD (US)

(73) Assignee: Futrex, Inc., Hagerstown, MD (US)

(21) Appl. No.: 13/009,654

(22) Filed: Jan. 19, 2011

Related U.S. Application Data
(60) Provisional application No. 61/296,331, filed on Jan. 19, 2010.

(51) Int. Cl. A61B 6/00

(52) U.S. Cl. 600/473; 600/476

(57) ABSTRACT

Systems and methods for measuring fat content of a body are provided. An instrument may be employed that generates light with different center wavelengths without the use of narrow optical band-pass filters and without the use of any light diffusing material. The instrument may include at least two different center wavelengths infrared emitting diodes (IREDs) having center wavelengths that are about 10 nanometers apart. A first IRED may have a center wavelength between 935 and 945 nanometers, and a second IRED may have a center wavelength between 945 and 955 nanometers. The IREDs may be arranged in a circular pattern in holes in an opaque medium. A near-infrared optical detector may be located at the center of the circular pattern. The instrument may perform a body fat measurement at a fixed distance from the crease in the elbow towards the biceps of the arm. The instrument may instead perform the body fat measurement at a fixed distance from the elbow bone towards the triceps of the arm.

### CROSS-REFERENCE TO RELATED APPLICATIONS

The present application claims the benefit of priority to U.S. Provisional Application Ser. No. 61/296,331, filed on Jan. 19, 2010, the entire disclosure of which is incorporated herein by reference.

### BACKGROUND OF THE INVENTION

1. Field of the Invention

The present invention is generally directed to systems and methods for determining percent body fat.

2. Description of the Related Art

The use of quantitative near-infrared measurement for determining percent body fat is known. For example, U.S. Pat. No. 4,633,087 to Rosenthal et al. (the “'87 patent,” the entire contents of which are incorporated herein by reference) discloses how to make such a measurement using two different wavelengths. U.S. Pat. No. 4,850,365 to Rosenthal (the “'365 patent,” the entire contents of which are incorporated herein by reference) discloses making such a measurement using a single wavelength or two different wavelengths plus physical parameters. In addition, U.S. Pat. No. 4,928,014 to Rosenthal (the “'014 patent,” the entire contents of which are incorporated herein by reference) describes a lower cost method of determining percent body fat using a single wavelength. The low-cost approach described in the '014 patent is specifically aimed for use in the privacy of a person's home as opposed to measurement by a health care professional.

This technology was further advanced by the teachings of U.S. Pat. No. 6,134,458 to Rosenthal (the “'458 patent,” the entire contents of which are incorporated herein by reference), which explains how the accuracy of the quantitative near-infrared measurement could be improved through the use of four to six wavelengths. The additional accuracy provided by the '458 patent enabled near-infrared measurement having the same accuracy as the “gold standard” method of underwater weighing (also called “hydrostatic” testing). However, the instrument described in the '458 patent is relatively expensive with the typical cost being approximately $4,000. This relatively high cost limits this lab accurate method of non invasive measurement of percent body fat to professional applications, such as medical applications. It is too expensive for most consumers to purchase for use in the privacy of their home.

The method of the '014 patent allows a relatively low-cost (<$100) commercial instrument to be developed for use in the privacy of the home (e.g., the FUTREX-1100 offered by Futrex Inc. of Hagerstown, Md.). However, the low-cost method of the '014 patent provides limited accuracy that may be insufficient for professional applications.

Thus, there is a need for a low-cost method that will provide the same accuracy as more expensive methods, such as the approach taught in the '458 patent, used in professional units. The following describes such a low-cost, accurate, near-infrared instrument.

### SUMMARY OF THE INVENTION

Having recognized the need for a low-cost, lab accurate device and method for measuring percent body fat, the inventor diligently worked for a solution. Although no one else has been able to do so, the inventor persevered. The surprising result significantly advances the state of the art by realizing an accurate device and method for measuring body fat that is also inexpensive and easy to use. Accurate measurement of percent body fat by consumers in the privacy of their homes is finally feasible.

According to an aspect of the invention, a method of determining percent body fat in the body may include steps of (a) transmitting near-infrared radiation into a body to achieve optical interactance between the body and the near-infrared radiation, (b) measuring optical absorption by the body at two or more wavelengths of the near-infrared radiation, and (c) utilizing the measured absorptions at each of the wavelengths of the near-infrared radiation to quantitatively determine fat content of the body. The transmitting, measuring and utilizing steps may not use narrow optical band-pass filters and do not use light diffusing material.

The near-infrared radiation may be within the range of 740-1100 nanometers. The two or more wavelengths may include first and second wavelengths at about 940 and 950 nanometers, respectively, and a third wavelength at about 810 nanometers. The third wavelength may instead be anywhere between 810 and 1100 nanometers except near the first and second wavelengths. The utilizing may utilize data on a plurality of physical parameters of the body along with the measured absorptions to quantitatively determine the fat content of the body. The physical parameters may be selected from a group consisting of height, weight, exercise level, sex, race, waist to hip measurement, arm circumference, and combinations thereof. The near-infrared radiation that is transmitted into the body may be from various point light sources located in a circle surrounding the optical detector which is mounted in opaque material. The method may further comprise using the opaque material to prevent light emitted by the light sources from being incident on the detector without first entering the body and being trans-reflected via interactance from the body. The method may further comprise using a digital weighing platform and providing a readout of both body fat and weight. The method may further comprise providing power for the transmitting, measuring and utilizing steps through a serial connection from the weighing platform. The method may further comprise providing power for the transmitting, measuring and utilizing steps from a battery. The transmitting may comprise sequentially transmitting near-infrared radiation into the body at different center wavelengths, and the measuring may comprise sequentially measuring the amount of light received from the body at each of the different center wavelengths.

According to another aspect of the invention, a method of determining percent body fat in the body may include steps of (a) transmitting near-infrared radiation to body to achieve optical interactance between the body and near-infrared radiation, (b) measuring optical absorptions of the near-infrared radiation by the body, and (c) quantitatively determining the fat content of the body using the measured absorptions of the near-infrared radiation in conjunction with data on a plurality of physical parameters of the body. The transmitting, measuring and determining steps do not use narrow optical band-pass filters and may not use light diffusing material.

The transmitting near-infrared radiation into the body may comprise emitting near-infrared radiation from several point light sources located in a circular pattern around an optical detector at the center of the circular pattern, and an opaque material may separate the optical detector from the light point sources. The instrument may further comprise using the opaque material to prevent light emitted by the light sources from being incident on the detector without first entering the body and being trans-reflected via interactance from the body. The determining may utilize data on a plurality of physical parameters of the body along with the measured absorptions to quantitatively determine the fat content of the body. The physical parameters may be selected from a group consisting of height, weight, exercise level, sex, race, waist to hip measurement, arm circumference, and combinations thereof. The near-infrared radiation may be within the range of 740-1100 nanometers. The measuring optical absorptions may comprise measuring the optical absorption of the near-infrared radiation at a plurality of different wavelengths. The measuring optical absorptions may comprise measuring the optical absorption of the radiation at two or more different wavelengths. One of the wavelengths may be about 940 nanometers+/−3 nanometers and the other of the wavelengths may be about 950 nanometers+/−3 nanometers with a minimum of about 10 nanometers between the two wavelengths. The method may further comprise using a digital weighing platform and providing a readout of both body fat and weight. The method may further comprise providing power for the transmitting, measuring and determining steps through a serial connection from the weighing platform. The method may further comprise providing power for the transmitting, measuring and determining steps from a battery. The transmitting may comprise sequentially transmitting near-infrared radiation into the body at different center wavelengths, and the measuring may comprise sequentially measuring the amount of light received from the body at each of the different center wavelengths.

According to an aspect of the invention, a near-infrared quantitative instrument for measuring fat content of a body is provided that may have an opaque medium, a plurality of infrared emitting diodes (IREDs) and a near-infrared optical detector. The plurality of IREDs may be arranged in a circular pattern in holes in the opaque medium, the plurality of IREDs having at least two different center wavelengths, which are about 10 nanometers apart, that include a center wavelength of between 935 and 945 nanometers and a center wavelength between 945 and 955 nanometers. The near-infrared optical detector may be located at the center of the circular pattern. The instrument does may not include narrow optical band-pass filters and does not include light diffusing material. The instrument may be configured to perform the body fat measurement at a fixed distance from the crease in the elbow of the body towards the biceps of the arm of the body. The instrument may be configured to perform the body fat measurement at a fixed distance from the elbow bone of the body towards the triceps of the arm of the body. The instrument may have a controller configured to cause the plurality of IREDs to sequentially illuminate the body and the detector to sequentially measure the amount of light received from the body at each of the different center wavelengths. The opaque material may configured to prevent light emitted by the IREDs from being incident on the detector without first entering the body and being trans-reflected via interactance from the body.

Further features and advantages of the present invention shall be understood in view of the following description with reference to the drawing figures.

### BRIEF DESCRIPTION OF THE DRAWINGS

The accompanying drawings, which are incorporated herein and form part of the specification, illustrate various embodiments of the present invention. In the drawings, like reference numbers indicate identical or functionally similar elements.

FIG. 1 provides the optical absorption of water and fat in the very near infrared spectrum region.

FIG. 2 compares body fat measurement accuracy between a conventional near-infrared instrument that uses the technology taught in the '458 patent, to an instrument that does not use narrow band pass optical filters in front of the infrared emitters.

FIG. 3 illustrates the measuring surface of a low cost, highly accurate percent body fat measurement system.

FIG. 4 compares the accuracy of an instrument based on the '458 patent to an instrument that uses four different infrared emitting diodes with neither optical filters nor a light diffuser spaced in a circle surrounding a detector that is mounted in optically opaque material.

FIG. 5 shows the statistical comparison of a commercial near-infrared body fat measurement instrument where measurement is made at the midpoint of the biceps to measurement made at different distances from the crease in the elbow in the direction towards the biceps.

FIG. 6 summarizes all the studies of FIG. 5.

FIG. 7 shows how a low-cost instrument using this invention is able to make a measurement at a fixed distance from the elbow.

FIG. 8 is a schematic illustration of an instrument for determining percent body fat according to one embodiment of the present invention.

### DETAILED DESCRIPTION OF THE PREFERRED EMBODIMENTS

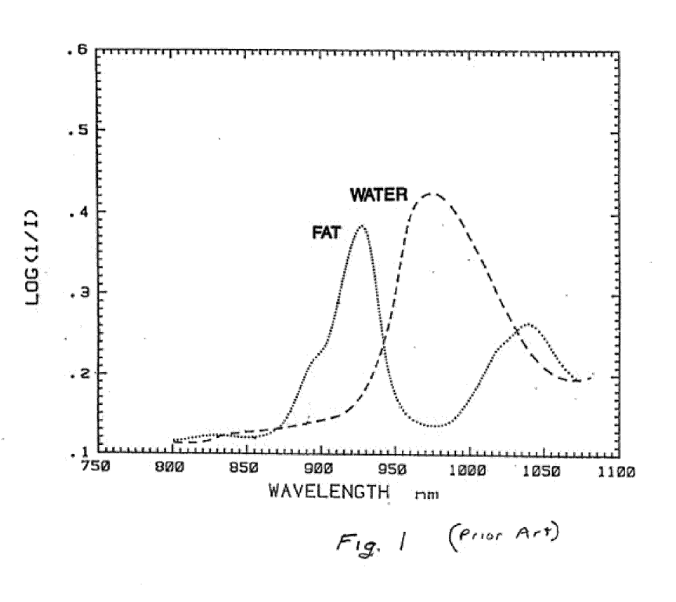

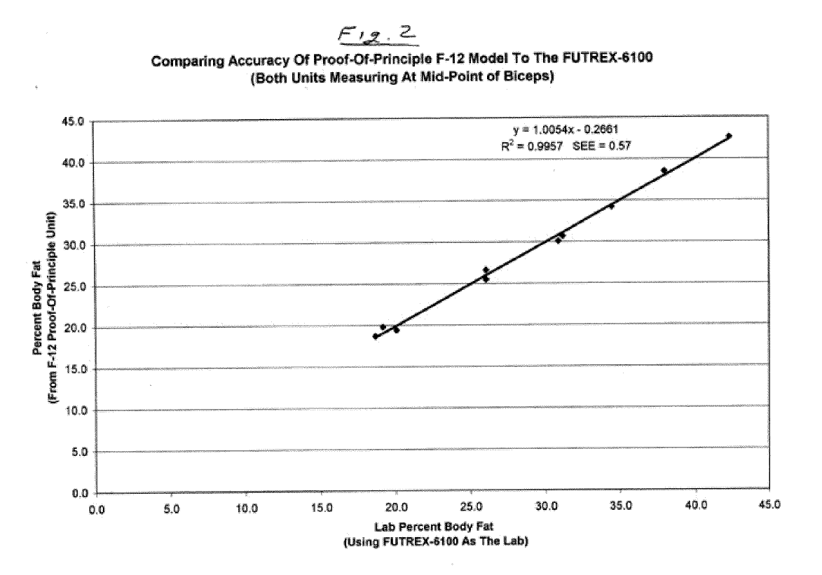

As shown in FIG. 1, the presence of water and the presence of fat have distinct optical signatures in the very near-infrared (NIR). The sensitivity to water starts rising at approximately 810 nm and peaks at approximately 970 nm. Following that peak, the absorbance due to water diminishes. The optical signature of fat also rises from approximately 810 nm to a peak at approximately 928 nm and then becomes less absorbent. In the region between 935 and 955 nm, the ratio of percent water to percent body fat has been shown in all mammals to be proportional to the percent body fat. This proportionality is the basis for the methods of the '365 and '458 patents.

Those patents teach the use of two or more infrared emitting diodes (IREDs) to perform the percent body fat measurement. In its simplest implementation, one of the IREDs has its center wavelength at the lower range of interest, between 935 to 945 nm, and another IRED has its center wavelength at a longer wavelength, between 950 to 955 nm. Since typical commercial IRED's have half power bandwidths of approximately 30 nm, measurement ability is sharpened by including in front of each IRED a narrow bandwidth optical filter, typically with a half power bandwidth of 10 nm.

In addition to the spectrum analysis described above, the '014 patent describes an alternate method of determining percent body fat. This method uses a single wavelength of light to effectively measure the “hardness” of the arm. The method operates on the premise that people with low body fat have harder arms that make it more difficult for light to penetrate the arm (i.e., the more physically fit the individual is, the lower the person's percent body fat is). The measurement is made by placing an illuminator and a detector, which is located approximately 0.5 inches from the illuminator, near the center of the biceps. The illuminator, which is a 950 nm IRED, is used as to emit light, and how much light is returned to a detector is measured. The more light is captured by the detector, the lower percent body fat is.

The present invention obtains essentially identical accuracy to that provided by the method of the '458 patent in a low-cost manner that takes advantage of both spectrum analysis and regression analysis. In an exemplary embodiment, spectrum analysis may be performed using commercially available IREDs with center wavelengths at approximately 940 nm and 950 nm. For instance, the Marubeni America Corporation, Part L940, and the Panasonic-SSG, Part LNA2802L, are examples of commercially available IREDs with center wavelengths at approximately 940 nm and 950 nm respectively. Additionally, the spectrum analysis may be performed without using any narrow band pass optical filters while still achieving the desired accuracy. Also, regression analysis may be multi-regression analysis that performs the “arm hardness” measurement. The multi-regression analysis may use, for example, an 810 nm IRED.

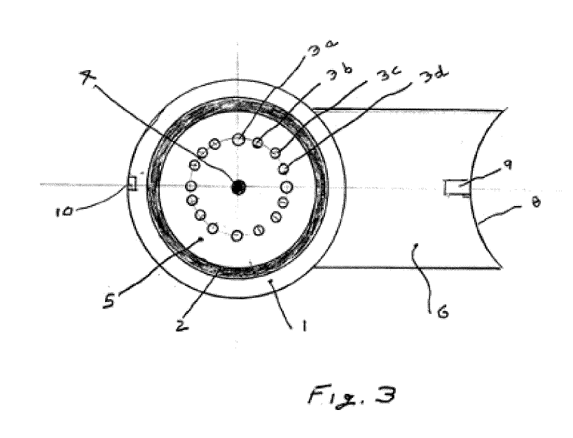

The test data, summarized in FIG. 2, illustrate that the accuracy obtained is essentially identical to the FUTREX-6100 instrument (Futrex, Inc., Hagerstown Md.). The FUTREX-6100 instrument uses the approach taught in the '458 patent and includes narrow band pass optical filters located in front of each IRED emitter. The ability to obtain the desired accuracy without the use of narrow band pass optical filters is important because narrow band pass optical filters are expensive. They are expensive to fabricate, and a housing must be provided to install them in front of the IREDs. Thus, by eliminating the need for narrow band optical filters, significant cost savings are obtained without degrading the measurement accuracy.

Moreover, in the '365 and '458 patents, the light generated by the IREDs and narrow band pass filters was then passed through a highly diffusing material to provide a uniform circular light pattern. Although such diffusing type material is not expensive, mounting it in a rigid, consistent fashion in an instrument is costly. Thus, by eliminating the need for mounting a highly diffusing material, significant cost savings are obtained without degrading the measurement accuracy.

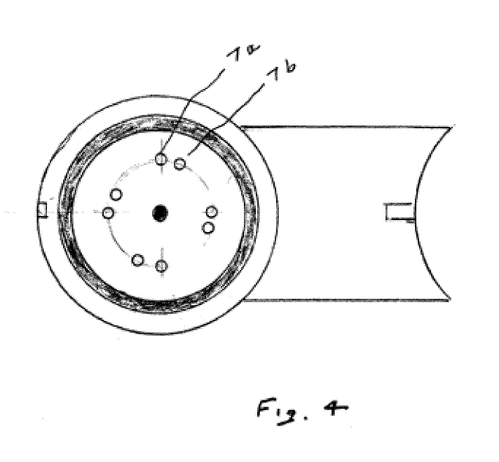

FIG. 3 illustrates the measuring surface 1 of an exemplary embodiment of an instrument in accordance with the present invention. The instrument may use four different IREDs 3a, 3b, 3c and 3d without optical narrow band pass filters and without any light diffuser. The four different IREDs 3a-3d may each have a different center wavelength and may be located in holes in an optically opaque material 5. The holes may form a circle surrounding an optical detector 4 located at the center of optically opaque material 5. Each of the four different center wavelength IREDs 3a-3d may be located in each 90 degree quadrant of the illumination circle. The center wavelengths of IREDs 3a-3d may be, for example, 810, 932, 940 and 950 nm respectively. In operation, light emitted from one of the IREDs 3a-3d enters into the body. Optical detector 4 captures light that has entered into the body and then been trans-reflected via interactance from the body to the optical detector 4. Each group of the same center wavelength IREDs 3a-3d may be sequentially illuminated to allow the optical detector 4 to sequentially measure the amount of light at each of the different wavelengths that has been trans-reflected to the optical detector 4. The energy captured from the optical detector 4 is then amplified, digitized and processed by a microprocessor to display the percent body fat (these standard electronic elements are not shown in the figure). Further, in this embodiment, an optically opaque soft foam material 2 prevents ambient light from interfering with the measurement. In this embodiment, no narrow band optical filters or light diffusers are used.

The measurement surface 1 from the crease in the elbow may be located by a rigid spacer 6. Edge 8 of the spacer 6 can be located at the elbow crease by positioning the guideline 9 at the center of the elbow crease.

An alternative embodiment is illustrated in FIG. 4. In this embodiment, two IRED parts 7a, 7b are used. Each part 7a, 7b may contain two separate, center wavelength IREDs. Each of the four IREDs of parts 7a and 7b may have a different center wavelength than the other IREDs of parts 7a and 7b. The center wavelengths of IREDs of parts 7a and 7b may be, for example, 810, 932, 940 and 950 nm respectively. As a result, the IREDs of parts 7a and 7b emit light can be separately and alternately illuminated by an electronic system to provide the same sequential four wavelength measurements used in the embodiment shown in FIG. 3. In this embodiment, no narrow band optical filters or light diffusers are used.

In either of the embodiments shown in FIGS. 3 and 4, the instrument may perform spectroscopic measurement, “hardness” measurement, and multi-regression analysis using certain physical parameters (e.g., age, sex, height, weight). The spectroscopic measurement, “hardness” measurement and physical parameters may be used simultaneously in a multiple linear regression equation to determine the percent body fat. As shown in the FIG. 2, the resulting accuracy is well within the inherent accuracy of the official method, underwater weighing (usually stated as 3.0%)

Thus, in accordance with the present invention, IREDs with center wavelengths of about 940 and 950 nm, or IRED's with center wavelengths of about 810, 932, 940 and 950 nm, may be used without narrow band optical filters and without a light diffuser to provide accuracy essentially equivalent to the accuracy produced using the teachings of the '458 patent.

Another requirement of previous near-infrared instruments is that the measurement be performed at the midpoint of the biceps. Research at the U.S. Department of Agriculture (USDA) has shown that the local percent body fat at this point is proportional to the total body's percent body fat. This USDA research also showed that the measurement can be made at the midpoint of the triceps. Although the midpoint of the triceps may be inconvenient for personal measurement, it can be of significant value in kiosk applications where an automatic measurement at the triceps may be preferred.

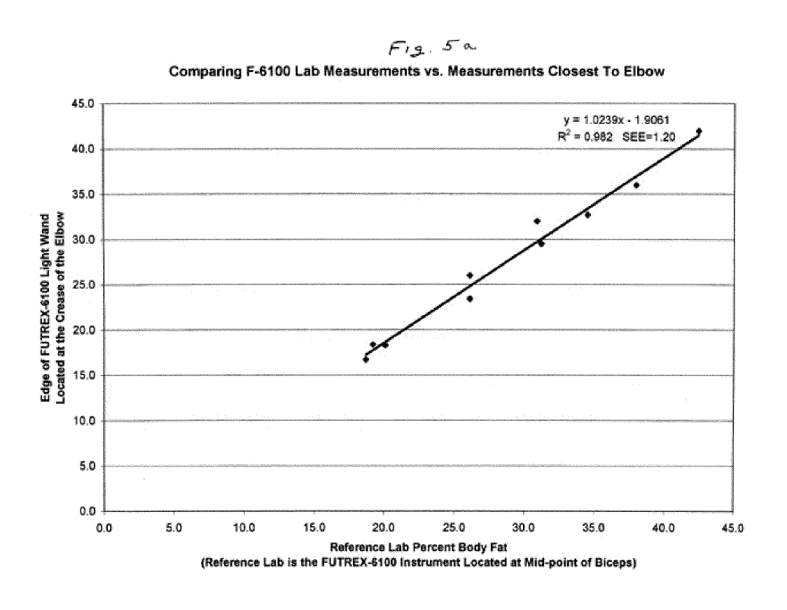

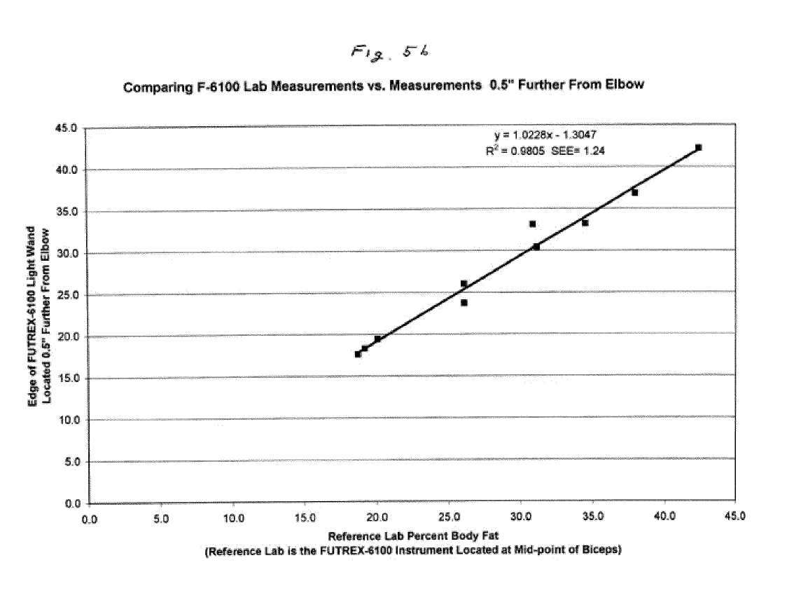

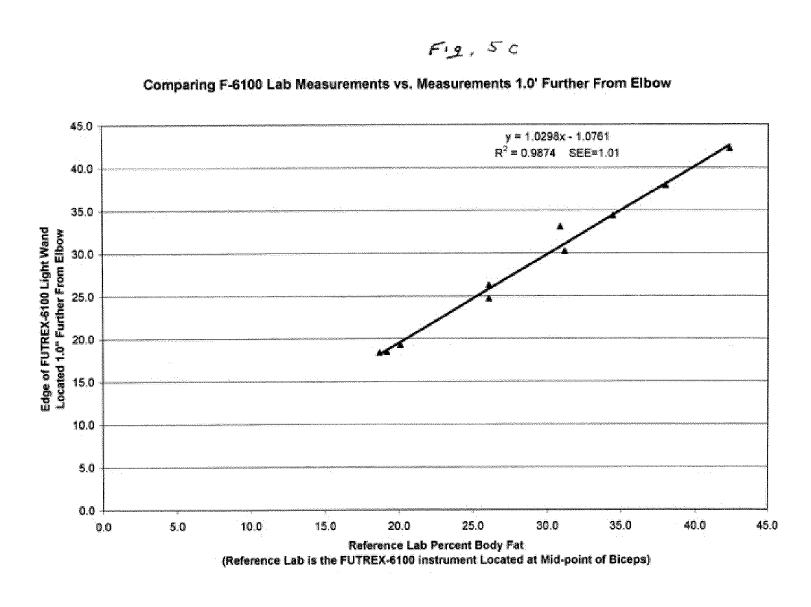

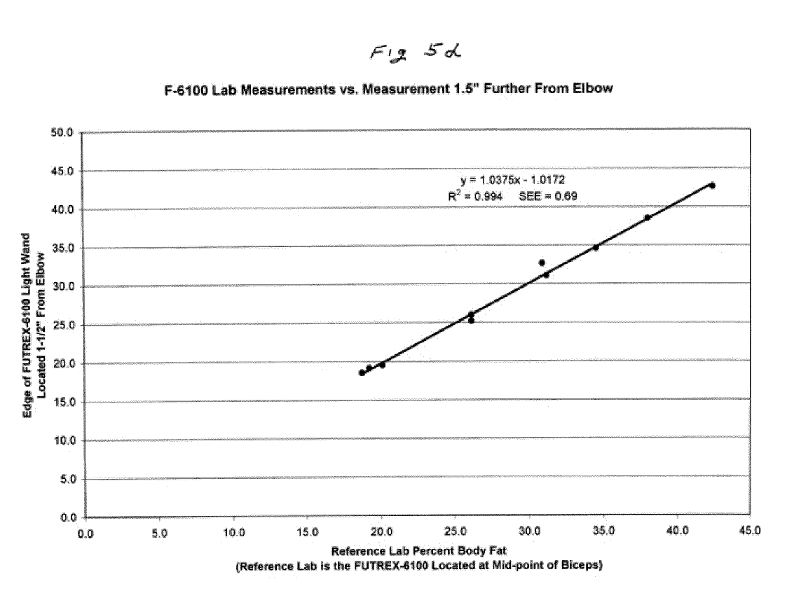

The FUTREX-6100 is a commercial instrument that uses the teaching of the '458 patent to provide accuracy equivalent to official under water weighing method for determining percent body fat. That instrument uses a “Light Wand” that has a diameter of 2 and ¼ inches. FIG. 5 shows the statistics of comparing “lab measurements” (i.e., measurements made at the midpoint of the biceps) of the FUTREX-6100 to measurements of the Futrex-6100 made at different distances from the crease in the elbow in the direction toward the biceps. FIG. 5a compares of the results of measuring ten, randomly selected volunteers with one edge of the Light Wand actually at the crease of the elbow to the lab measurements made at the midpoint of the biceps. As shown in the FIG. 5a, the measurements at the crease of the elbow result in a coefficient of determination (R-squared or R2) of 0.982 and a Standard Error of Estimate (SEE) of 1.20.

The same ten volunteers were then re-measured after the Light Wand was moved a half inch from the crease of the elbow towards the shoulder, and the measurements were compared to the lab measurements made at the midpoint of the biceps (FIG. 5b). In this case, the SEE slightly increased to 1.24. This approach was repeated with measurements taken with the Light Want located 1.0 inch from the elbow crease (FIG. 5c), with measurements taken with the Light Wand located 1.5 inches from the elbow crease (FIG. 5d), and with measurements taken with the Light Wand located 2.0 inches from the elbow crease (FIG. 5e).

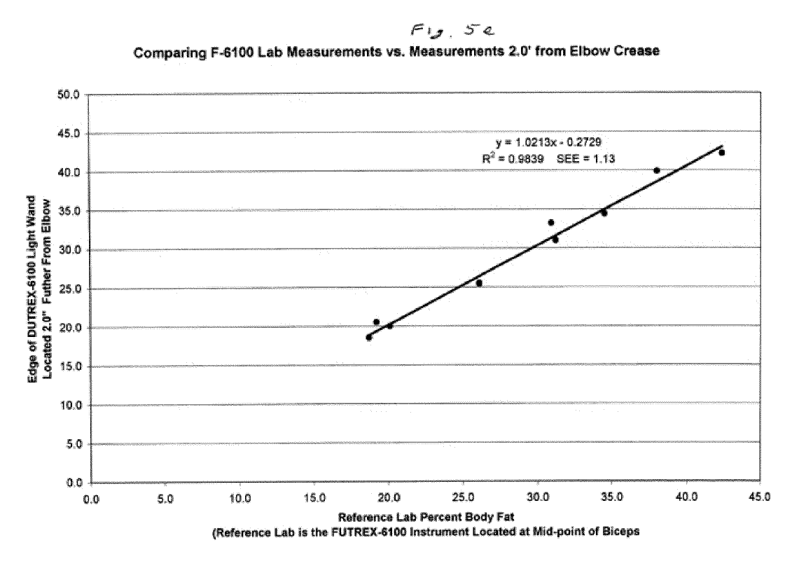

FIG. 6 provides a summary of all the studies shown in FIG. 5. As shown, the best results occurred when the Light Wand was located at 2.6 inches (i.e., the 1.1 inch radius of the Light Wand plus 1.5 inches) from the crease of the elbow in the direction of the shoulder and centered on the biceps side of the arm. The resultant SEE is approximately 0.7 and well within the accuracy of the official method. Therefore, the center of the measuring surface of an instrument in accordance with the present invention will preferably be located 2.6 inches from the crease of the elbow in the direction of the shoulder and centered on the biceps side of the arm.

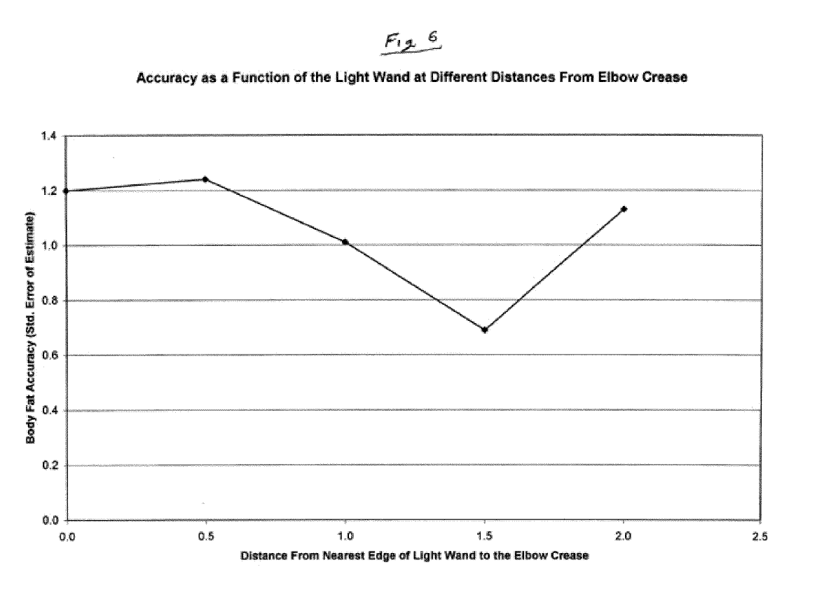

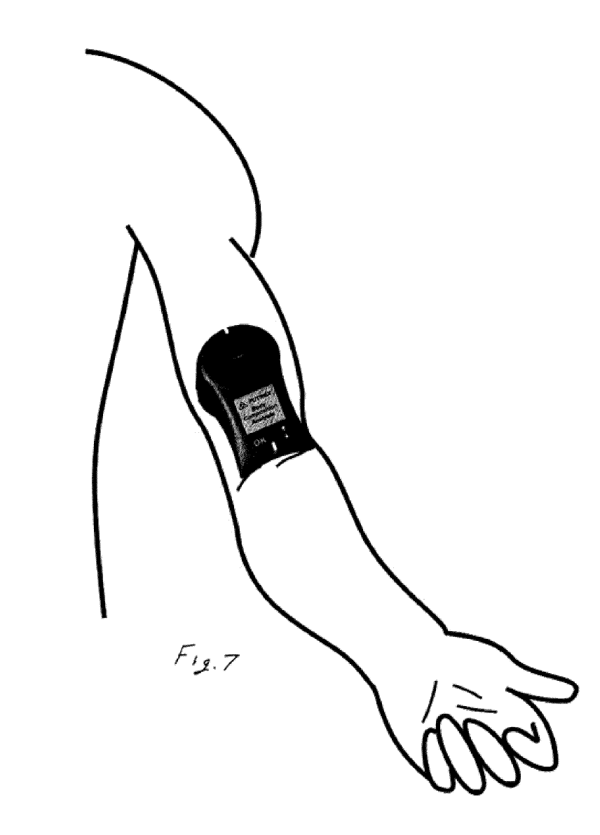

FIG. 7 illustrates an exemplary conceptual design of a low-cost instrument in accordance with the present invention. By having one edge at the crease of the elbow as shown, the center of the measuring surface is properly positioned, and the user does not have to locate the midpoint of the biceps to make the measurement. Thus, use of the instrument by consumers in the privacy of their home is made easier by the elimination of the need for a difficult measurement location.

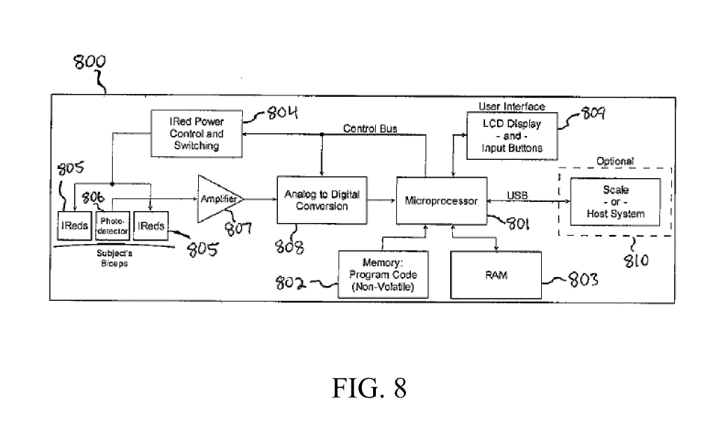

FIG. 8 is a schematic illustration of an instrument 800 for determining percent body fat according to an embodiment of the present invention. The schematic illustration shown in FIG. 8 may, for example, be used with either of the instruments shown in FIGS. 3 and 4.

Instrument 800 may include a microprocessor 801. Microprocessor 801 may execute a firmware program code stored in a non-volatile memory 802, such as a read only memory (ROM). Microprocessor 801 may use a memory 803, such as a random access memory (RAM), during the course of measurement, calculation and display of percent body fat results.

Microprocessor 801 may be coupled to an IRED power control and switching unit 804. Per instructions from microprocessor 801, IRED power control and switching unit 804 may control the power of IREDs 805, which may correspond to IREDS 3a-3d of FIG. 3 or the IREDs of IRED parts 7a and 7b of FIG. 4. In addition, microprocessor 801 may control the timing of the switching of IREDs 805 through IRED power control and switching unit 804.

The IREDs 805 may provide illumination for the arm of a subject. The IREDs may provide two or more different wavelengths of near-infrared light and may be turned on and off by the IRED power control and switching unit 804 under the control of the microprocessor 801.

The near-infrared light emitted by IREDs 805 may enter the biceps, and the levels of the different wavelengths of the near-infrared light may impinge on a photodetector 806 after entering the bicep and bouncing back. Photodetector 806 may be a silicon photodiode. Photodetector 806 may correspond to the optical detector 4 of FIGS. 3 and/or 4. The levels of the different wavelengths of the near-infrared light that impinge on a photodetector 806 may be converted to electric currents that are used by microprocessor 801 to calculate percent body fat.

Instrument 800 may also include an amplifier 807 coupled to the photodetector 806. Amplifier 807 may be configured in a transconductance mode and may provide variable voltages corresponding to the electric currents output from the photodetector 806. The voltages output from the amplifier 807 may be supplied to an analog-to-digital converter (ND) 808. ND 808 may digitize the signals from the amplifier and output the digitized signals to microprocessor 801 for analysis.

Instrument 800 may include a user interface 809 that may include an LCD display and/or several input keys. The LCD display and/or several input keys of user interface 809 may be coupled to the microprocessor 801. The display and keys of the user interface 809 may enable a user to enter data, start the percent body fat reading and view the results.

Instrument 800 may also include a USB port or interface through which optional devices 810 may be connected to microprocessor 801. For example, optional devices 810 may include a digital scale, and the built in USB interface enables instrument 800 to be combined with the digital scale for providing simultaneous measurements of weight and percent body fat. Alternatively, or in addition, a host device may be connected to microprocessor 801 through the USB port and communicate and/or control the instrument 800 through the USB port. Moreover, the instrument could operate off of the voltage and current supplied by the USB interface, and, as a result, the instrument would not require either a battery or an AC adapter. Alternatively, a battery may be installed in the instrument to allow the instrument to be used without a digital scale in the privacy of the home.

While the invention has been disclosed in detail above, the invention is not intended to be limited to the invention as disclosed. It is evident that those skilled in the art may now make numerous uses and modifications of and departures from the specific embodiments described herein without departing from the inventive concepts.

While various embodiments/variations of the present invention have been described above, it should be understood that they have been presented by way of example only, and not limitation. Thus, the breadth and scope of the present invention should not be limited by any of the above-described exemplary embodiments. Further, unless stated, none of the above embodiments are mutually exclusive. Thus, the present invention may include any combinations and/or integrations of the features of the various embodiments.

Additionally, while the processes described above and illustrated in the drawings are shown as a sequence of steps, this was done solely for the sake of illustration. Accordingly, it is contemplated that some steps may be added, some steps may be omitted, and the order of the steps may be re-arranged.

We claim:

1. A method of determining percent body fat in the body, comprising:
(a) transmitting near-infrared radiation into a body to achieve optical interactance between the body and the near-infrared radiation,
(b) measuring optical absorption by the body at two or more wavelengths of said near-infrared radiation, and
(c) utilizing the measured absorptions at each of the wavelengths of the near-infrared radiation to quantitatively determine fat content of the body;
wherein the transmitting, measuring and utilizing steps do not use narrow optical band-pass filters and do not use light diffusing material.

2. The method of claim 1, wherein said near-infrared radiation is within the range of 740-1100 nanometers.

3. The method of claim 1, wherein the two or more wavelengths comprise two of the wavelengths at about 940 and 950 nanometers, respectively, and a third wavelength at about 810 nanometers or anywhere between 810 and 1100 nanometers except near the wavelengths of about 940 and 950 nanometers, respectively.

4. The method of claim 1, wherein the utilizing step utilizes data on a plurality of physical parameters of the body along with said measured absorptions to quantitatively determine the fat content of the body.

5. The method of claim 4, wherein said physical parameters are selected from a group consisting of height, weight, exercise level, sex, race, waist to hip measurement, arm circumference, and combinations thereof.

6. The method of claim 1, wherein the near-infrared radiation that is transmitted into the body is from various point light sources located in a circle surrounding the optical detector which is mounted in opaque material to prevent light emitted by the light sources from being incident on the detector without first entering the body and being trans-reflected via interactance from the body.

7. The method of claim 1, further comprising using a digital weighing platform and providing a readout of both body fat and weight.

8. The method of claim 1, wherein the transmitting step comprises sequentially transmitting near-infrared radiation into the body at different center wavelengths; and
the measuring comprises sequentially measuring the amount of light received from the body at each of the different center wavelengths.

9. A method of determining percent body fat in the body, comprising:
(a) transmitting near-infrared radiation to body to achieve optical interactance between the body and near-infrared radiation,
(b) measuring optical absorptions of said near-infrared radiation by the body, and
(c) quantitatively determining the fat content of the body using the measured absorptions of said near-infrared radiation in conjunction with data on a plurality of physical parameters of the body;
wherein the transmitting, measuring and determining steps do not use narrow optical band-pass filters and do not use light diffusing material.

10. The method of claim 9, wherein the transmitting near-infrared radiation into said body step comprises emitting near-infrared radiation from several point light sources located in a circular pattern around an optical detector at the center of the circular pattern, and an opaque material separates the optical detector from the light point sources, to prevent light emitted by the light sources from being incident on the detector without first entering the body and being trans-reflected via interactance from the body.

11. The method of claim 9, wherein the determining step utilizes data on a plurality of physical parameters of the body along with said measured absorptions to quantitatively determine the fat content of the body.

12. The method of claim 11, wherein said physical parameters are selected from a group consisting of height, weight, exercise level, sex, race, waist to hip measurement, arm circumference, and combinations thereof.

13. The method of claim 9, wherein said measuring optical absorptions step comprises measuring the optical absorption of said near-infrared radiation at a plurality of different wavelengths.

14. The method of claim 13, wherein one of said wavelengths is about 940 nanometers+/−3 nanometers and another of said wavelengths is about 950 nanometers+/−3 nanometers with a minimum of about 10 nanometers between said wavelengths.

15. The method of claim 9, wherein the transmitting step comprises sequentially transmitting near-infrared radiation into the body at different center wavelengths; and
the measuring comprises sequentially measuring the amount of light received from the body at each of the different center wavelengths.

16. A near-infrared quantitative instrument for measuring fat content of a body, the instrument comprising:
an opaque medium;
a plurality of infrared emitting diodes (IREDs) arranged in a circular pattern in holes in the opaque medium, the plurality of IREDs having at least two different center wavelengths, which are about 10 nanometers apart, that include a center wavelength of between 935 and 945 nanometers and a center wavelength between 945 and 955 nanometers; and
a near-infrared optical detector located at the center of the circular pattern;
wherein the instrument does not include narrow optical band-pass filters and does not include light diffusing material.

17. The instrument in claim 16, wherein the instrument is configured to perform the body fat measurement at a fixed distance from the crease in the elbow of the body towards the biceps of the arm of the body.

18. The instrument of claim 16, wherein the instrument is configured to perform the body fat measurement at a fixed distance from the elbow bone of the body towards the triceps of the arm of the body.

19. The instrument of claim 16, further comprising a controller configured to cause the plurality of IREDs to sequentially illuminate the body and the detector to sequentially measure the amount of light received from the body at each of the different center wavelengths.

20. The instrument of claim 16, wherein the opaque material is configured to prevent light emitted by the IREDs from being incident on the detector without first entering the body and being trans-reflected via interactance from the body.
